(19) United States
(12) Patent Application Publication
(10) Pub. No.: US 2012/0266386 A1
(43) Pub. Date: Oct. 25, 2012

(54) Water-Soluble Pouches Containing Bleaching Agents

(76) Inventor: Jonathan Propper, Haverford, PA (US)

(21) Appl. No.: 13/446,389

(22) Filed: Apr. 13, 2012

Related U.S. Application Data
(60) Provisional application No. 61/478,565, filed on Apr. 25, 2011.

(51) Int. Cl. B65D 69/00; A61L 2/18; D06L 3/16

(52) U.S. Cl. 8/108.1; 8/101; 422/29; 206/223

(57) ABSTRACT

Water-soluble pouches containing a bleaching agent are provided. Also provided are methods for using such pouches for bleaching or disinfecting laundry or surfaces.

### CROSS REFERENCE TO RELATED APPLICATIONS

This application claims priority to U.S. Provisional Application No. 61/478,565, filed on Apr. 25, 2011, the contents of which are incorporated by reference herein, in their entirety and for all purposes.

### FIELD OF THE INVENTION

The invention relates generally to household cleaning supplies. More particularly, the invention relates to water-soluble pouches containing a pre-measured liquid bleach solution that can be added to water in order to create a bleaching solution for bleaching or disinfecting materials, including laundry, as well as kits containing such pouches and methods for using them.

### BACKGROUND OF THE INVENTION

Various publications, including patents, published patent applications, technical articles, and scholarly articles, are cited throughout the specification. Each of these cited publications is incorporated by reference in this document, in its entirety and for all purposes.

Chlorine-based bleach is used in many households as an aid to whitening clothing and fabrics during laundry. In addition, chlorine-based bleach may be used as a disinfectant in other cleaning applications, such as cleaning dishes in dishwashers, cleaning toilets, and removing mold and mildew from bathtubs and shower stalls, among others. But chlorine-based bleach is a hazardous material that must be diluted in large amounts of water so that it can be handled more safely. In addition, accidental spills of chlorine-based bleach may cause permanent damage to clothing, carpets, or other materials that come in contact with the spilled bleach. And, spilled bleach emits a noxious chlorine odor.

Peroxide-based bleach is an alternative to chlorine-based bleach. Peroxide-based bleach is also used in household cleaning applications, most commonly in laundry. Although peroxide-based bleach is less damaging to colored materials, and emits significantly less noxious odors, it is still corrosive, and may cause serious injuries to skin or eyes in the event of accidental contact.

To reduce the need for handling bleach agents, to reduce accidental spills of bleaching agents, and to reduce accidental contact with bleaching agents, a need exists for consumer-friendly containers for bleaching agents.

### SUMMARY OF THE INVENTION

The invention features pouches comprising a body comprising a water-soluble material and defining a container portion, and a bleaching agent or composition comprising a bleaching agent contained within the container portion. The container portion preferably has a single layer having an internal surface contacting the bleaching agent or composition and an external surface comprising the outermost portion of the pouch, although the container portion may have more than one layer, such as an outer layer, which may be a coating. In some preferred aspects, the pouch has no external coating. The bleaching agent or composition thereof preferably has less than about 10% water, less than about 7% water, less than about 5% water, or less than about 3% water, according to the weight of the bleaching agent or composition. The pouch preferably does not contain any cleaning agents such as detergents, and is made from polyvinyl alcohol.

In preferred aspects, the pouch comprises a composition comprising a bleaching agent contained within the container portion. The composition comprises a carrier comprising a hydrophobic vehicle such as an alcohol, which may be a polyol such as a glycol, and preferably hexylene glycol. The composition preferably does not contain any cleaning agent such as a detergent. The bleaching agent may be a chlorine-based bleaching agent, or an oxidative bleaching agent, which may be chlorine-free.

The composition may comprise, by weight of the composition, about 0.5% to about 1.5% of a chlorine-based bleaching agent, about 6% to about 8% water, and about 90% to about 95% of a carrier, or about 0.8% to about 1.1% of a chlorine-based bleaching agent, about 6.5% to about 7.5% water, and about 91% to about 93% of a carrier, or about 0.9% of a chlorine-based bleaching agent, about 7% water, and about 92% of a carrier. The chlorine-based bleaching agent may be selected from the group consisting of sodium hypochlorite, calcium hypochlorite, lithium hypochlorite, chlorinated isocyanurates, isocyanuric acid, dichlorodimethyl hydantoin, chlorinated phosphates, and chloramines. The carrier may be a polyol, including glycols such as hexylene glycol. Such compositions, free of a pouch, are within the scope of the invention.

The composition may comprise, by weight of the composition, about 2% to about 4% of an oxidative bleaching agent, about 5% to about 7% water, and about 88% to about 92% of a carrier, about 3% to about 4% of an oxidative bleaching agent, about 6% to about 7% water, and about 89% to about 91% of a carrier, or about 3.5% of an oxidative bleaching agent, about 6.5% water, and about 90% of a carrier. The oxidative bleaching agents may be selected from the group consisting of crystalline sodium perborate, sodium percarbonate, sodium perphosphate, percarbamide, sodium persulfate, tetrasodium pyrophosphate, tetraacetylethylenediamine, sodium nonanoyloxybenzenesulfonate, calcium peroxide, zinc peroxide, sodium peroxide, carbamide peroxide, chlorine dioxide, bromate, and benzoyl peroxide. The carrier may be a polyol, including glycols such as hexylene glycol. Such compositions, free of a pouch, are within the scope of the invention.

The invention also features laundry kits comprising one or more pouches comprising a body comprising a water-soluble material and defining a container portion, a bleaching agent or composition comprising a bleaching agent contained within the container portion, and instructions for using the kit in a method for bleaching laundry, including a method for cleaning and bleaching laundry. The kits may comprise any pouch described or exemplified herein. The kits may include instructions for using the one or more pouches in a high efficiency wash cycle, or for using the one or more pouches in a regular wash cycle. The kits may further comprise one or more cleaning agents, one or more fabric softening agents, or one or more fragrance, aroma, essential oil, or composition thereof.

The invention also features methods for bleaching a material or for disinfecting a material, which may be a surface (which need not comprise a fabric), or which may be laundry, comprising adding a water-soluble pouch containing a bleaching agent or a composition comprising a bleaching agent to a volume of water such that the pouch dissolves in the water thereby creating a bleach water solution, and contacting material with the bleach water solution for a period of time sufficient to bleach the material or for a period of time sufficient to disinfect the material.

The invention also features methods for cleaning laundry, comprising contacting the laundry with a volume of water comprising one or more cleaning agents for a period of time sufficient to clean the laundry, optionally rinsing the laundry to remove the one or more cleaning agents from the laundry, adding a water-soluble pouch containing a bleaching agent or composition comprising a bleaching agent to the water such that the pouch dissolves in the water thereby creating a bleach water solution, and contacting the laundry with the bleach water solution for a period of time sufficient to bleach the laundry. The methods may further comprise rinsing the laundry to remove the bleach water solution from the laundry. The methods may further comprise drying the laundry.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided in this document.

As used herein, the singular forms “a,” “an,” and “the” include plural referents unless expressly stated otherwise.

Laundry includes, but is not limited to, any materials to be washed or cleaned, including articles of clothing, sheets and linens, blankets, tapestries, napkins, upholstery, towels, curtains, area rugs, stuffed animals, pillows, footware, fabrics, and other fabric-based materials.

The invention features water-soluble pouches containing a bleaching agent such as a chlorine-based or peroxide-based bleaching agent. Additionally, the invention features methods for bleaching materials using these pouches. The pouches and methods may find use in various cleaning applications, for example, for bleaching laundry, or for bleaching and disinfecting a surface or material. The pouches and methods may be used in household, commercial, or industrial cleaning applications.

The pouch may be fabricated out of any suitable material, and is preferably fabricated out of a water-soluble material. Thus, the pouch is preferably water soluble. Non-limiting examples of suitable materials for fabricating the pouch include polyvinyl alcohol, partially hydrolyzed polyvinyl acetate, and alginates, and may take the form of a film. The materials for the pouch may be formulated to particular water-solubility specifications, including partial or complete solubility in cold, lukewarm, warm, hot, or very hot water. The pouch preferably has a single layer of suitable materials, with the internal surface of the layer in contact with the contents of the pouch and with the external surface of the layer comprising the outermost surface of the pouch. The pouch may also have multiple layers, for example, two layers. The outer layer or layers may be a coating, and may allow the pouch to maintain its structural integrity for longer periods of time. In some preferred aspects, the pouch does not have a coating. The pouch may be manufactured in any suitable shape, including a substantially round, cylindrical, or rectangular shape.

The pouch material preferably can withstand the water content of liquids contained within the pouch, for example bleaching agents or compositions comprising bleaching agents. Preferably, the pouch material can withstand water content of about 10% or less, about 7% or less, about 5% or less, or about 2% or less in the bleaching agents or compositions comprising bleaching agents contained in the pouch when stored over time, yet the pouch readily dissolves when immersed in water or aqueous cleaning solutions.

The water solubility of the pouch, including the extent and rate of dissolution, may relate, in part, to the thickness of the pouch film. The thickness may be a function of the amount of the materials used, or the number of layers that make up the pouch. The thickness of the pouch should impart a degree of mechanical strength to the pouch such that it can be handled by a user without rupturing, leaking, or dissolving in the user's hand. The thickness of the pouch, particularly the container portion of the pouch, may range from about 0.1 to about 2 mm, although smaller or larger thicknesses are possible. Preferred thicknesses may be about 0.2 mm, about 0.3 mm, about 0.5 mm, or about 1 mm. In some preferred aspects, the pouch 10 is not coated with any additional materials.

In some preferred aspects, the pouch contains a pre-determined volume of a bleaching agent or a composition comprising a bleaching agent. For example, a pouch may be manufactured and provided to a consumer in a ready-to-use form, and the pouch may take the form of a bag, packet, or sachet. For laundry applications, the volume of the bleaching agent or composition comprising a bleaching agent contained within the pouch may be a volume suitable for a regular wash cycle, for a high efficiency wash cycle, for a large load cycle including load size for commercial laundromats. For general cleaning applications such as surface cleaning applications, the volume of the bleaching agent or composition comprising a bleaching agent contained within the pouch may be a volume suitable for adding to a small volume of water. Non-limiting examples of small volumes of water to which a pouch may be added include about 0.5 to about 5 gallons of water or aqueous cleaning solution in a bucket for household cleaning applications, or about 0.1 to about 0.5 gallons of water or aqueous cleaning solution in a small spray bottle or squirt bottle for household cleaning applications. A ready-to-use pouch preferably does not contain any cleaning agent such as a detergent.

A pouch may be formed, for example, from one or more sheets of a packaging material or from a tubular section of such material. In some aspects, the pouch may be formed from a single folded sheet of material or from two sheets of material, sealed together at the edges, for example, with an adhesive or by heat-sealing. The pouch may be prepared according to U.S. Pat. No. 6,037,319, including the materials and specifications set forth in that patent.

The inventive pouches are suitable for use with any bleaching agent or composition comprising a bleaching agent. The bleaching agent may be a chlorine-based bleaching agent, or may be a chlorine-free bleaching agent, including oxidative bleaching agents. Non-limiting examples of chlorine-based bleaching agents include sodium hypochlorite, calcium hypochlorite, lithium hypochlorite, chlorinated isocyanurates, isocyanuric acid, dichlorodimethyl hydantoin, chlorinated phosphates, and chloramines. Non-limiting examples of chlorine-free and oxidative bleaching agents include crystalline sodium perborate (sodium peroxyborate tetrahydrate), sodium percarbonate, sodium perphosphate, percarbamide, sodium persulfate, tetrasodium pyrophosphate, urea peroxide plus catalysts and activators, e.g., tetraacetylethylenediamine or sodium nonanoyloxybenzenesulfonate, calcium peroxide, zinc peroxide, sodium peroxide, carbamide peroxide, chlorine dioxide, bromate, and benzoyl peroxide. The chlorine-based or oxidative bleaching agent may be a solid or may be a liquid, for example, an aqueous solution of a bleaching agent.

A bleaching agent composition may comprise, in addition to the bleaching agent, a suitable carrier. The carrier may comprise a suitable hydrophobic vehicle, including an organic solvent, oil, or alcohol such as ethanol, isopropanol, or a polyol. Non-limiting examples of suitable polyols include glycol such as ethylene glycol, polyethylene glycol, 1,2-propane diol, propylene glycol, butylene glycol, neopentyl glycol, and hexylene glycol, as well as glycerol, mannitol, maltitol, sorbitol, erythritol, xylitol, glucose, fructose, lactose and erythritan. The carrier may comprise an aqueous vehicle such as water. The composition may comprise an emulsion, including an oil-in-water or water-in-oil emulsion. Preferably, the composition does not include any detergent.

The composition may comprise a suitable amount of one or more surfactants, including anionic, cationic, zwitterionic, and nonionic surfactants, emulsifiers, suspending agents and combinations thereof. The composition may also comprise one or more buffering agents. The composition may comprise a dye to give the composition a desired color. The composition may have an acidic, basic, or neutral pH. A neutral pH is preferred.

The composition is preferably formulated to be compatible with the pouch, without compromising the integrity of the pouch, weakening the pouch, or dissolving the pouch over time during shelf storage. In the pouch, the composition is preferably contained within a single layer of material having an internal surface directly contacting the composition, and having an external surface which is the outside of the pouch.

In some aspects, a composition comprises about 0.25% to about 2.5% of a chlorine-based bleaching agent, about 4% to about 10% water, and about 85% to about 97% of a carrier; percentages according to the weight of the composition. The composition may comprise about 0.5% to about 1.5% of a chlorine-based bleaching agent, about 6% to about 8% water, and about 90% to about 95% of a carrier; percentages according to the weight of the composition. The composition may comprise about 0.8% to about 1.1% of a chlorine-based bleaching agent, about 6.5% to about 7.5% water, and about 91% to about 93% of a carrier. In some detailed aspects, the composition may comprise about 0.9% of a chlorine-based bleaching agent, about 7% water, and about 92% of a carrier; percentages according to the weight of the composition. For example, the composition may comprise about 92% by weight of a carrier and about 8% by weight of a blend of a water (about 88% of the blend) and chlorine-based bleaching agent (about 12% of the blend). The chlorine-based bleaching agent may comprise about 0.1%, about 0.25%, about 0.5%, about 0.75%, about 1%, about 1.25%, about 1.5%, about 1.75%, about 2%, about 2.25%, about 2.5%, about 2.75%, about 3%, about 4%, or about 5% by weight of the composition. Water may comprise about 2%, about 2.5%, about 3%, about 3.5%, about 4%, about 4.5%, about 5%, about 5.5%, about 6%, about 7%, about 8%, about 9%, or about 10% by weight of the composition. The carrier may be an alcohol carrier, and preferably a polyol carrier. A polyol carrier may be a glycol carrier, preferably ethylene glycol, propylene glycol, hexylene glycol, or combinations thereof. The composition preferably has a neutral pH.

In some aspects, a composition comprises about 1% to about 6% of an oxidative bleaching agent, about 4% to about 10% water, and about 85% to about 95% of a carrier; percentages according to the weight of the composition. The composition may comprise about 2% to about 4% of an oxidative bleaching agent, about 5% to about 7% water, and about 88% to about 92% of a carrier; percentages according to the weight of the composition. The composition may comprise about 3% to about 4% of an oxidative bleaching agent, about 6% to about 7% water, and about 89% to about 91% of a carrier. In some detailed aspects, the composition may comprise about 3.5% of an oxidative bleaching agent, about 6.5% water, and about 90% of a carrier; percentages according to the weight of the composition. For example, the composition may comprise about 90% by weight of a carrier and about 10% by weight of a blend of a water (about 65% of the blend) and oxidative bleaching agent (about 35% of the blend). The oxidative bleaching agent may comprise about about 0.5%, about 0.75%, about 1%, about 1.5%, about 2%, about 2.5%, about 3%, about 3.5%, about 4%, about 4.5%, about 5%, about 5.5%, about 6%, or about 6.5% by weight of the composition. Water may comprise about 2%, about 2.5%, about 3%, about 3.5%, about 4%, about 4.5%, about 5%, about 5.5%, about 6%, about 7%, about 8%, about 9%, or about 10% by weight of the composition. The carrier may be an alcohol carrier, and preferably a polyol carrier. A polyol carrier may be a glycol carrier, preferably ethylene glycol, propylene glycol, hexylene glycol, or combinations thereof. The composition preferably has a neutral pH. The composition may comprise a dye to give the composition a desired color.

It is preferable to limit the amount of water in the composition to less than 10% of the weight of the composition. In some aspects, it is preferable to limit the amount of water to less than about 8%, less than about 7% of the weight of the composition, less than about 6% of the weight of the composition, or less than about 5% of the weight of the composition. It is preferred that the pouch containing a composition is stable over extended periods of time of shelf storage, including at least about 6 months, at least about 9 months, at least about 12 months, at least about 15 months, at least about 18 months, or at least about 24 months, or longer. Shelf storage conditions preferably include room temperature, and low to moderate humidity.

The invention also features kits. The kits may be packaged in a suitable container, for example, a box or a case.

In some aspects, the kits comprise one or more water-soluble pouches, such as a pouch described or exemplified herein, containing a bleaching agent or composition comprising a bleaching agent and instructions for using the kit in a method for cleaning a material, including cleaning laundry. The bleaching agent composition may be any composition described or exemplified herein. The pouch preferably has a single layer, with the internal surface of the layer contacting the bleaching agent or composition thereof, and the external surface of the layer comprising the outer surface of the pouch. The kits may further include one or more cleaning agents such as a detergent. The kits may further include a fabric softening agent. Such cleaning agents and fabric softening agents may be provided in separate water-soluble pouches, and preferably are not contained in the same pouch that contains a bleaching agent or composition comprising a bleaching agent.

The invention also features methods. in some aspects, the methods may be used to bleach a material. Generally, the methods comprise adding a pouch containing a bleaching agent or a composition comprising a bleaching agent to a volume of water such that exposure of the pouch to the water for a sufficient period of time causes the pouch to dissolve in the water, thereby releasing the contents of the pouch into the water and creating a bleach water solution, and contacting a material with the bleach water solution for a period of time sufficient to bleach the material. In some preferred aspects, the water is in the drum of a washing machine, or is being added to the drum of a washing machine, for example, during a filling or rinse cycle. Preferably, the pouch does not contain any cleaning agent such as a detergent or a fabric softening agent. The pouch preferably has a single layer, with the internal surface of the layer contacting the bleaching agent or composition thereof, and the external surface of the layer comprising the outer surface of the pouch. The material may be any material, and in some preferred aspects is laundry. The bleach water solution may be in a spray bottle or squirt bottle which are used to contact the material with the bleach water solution.

In some aspects, the water comprises a cleaning agent, for example, a detergent such as a laundry detergent. In some aspects, the water comprises a fabric softening agent. The pouch may be added to the water substantially at the same time as the cleaning agent or the fabric softening agent, or may be added to the water before or after the cleaning agent or fabric softening agent is added to the water. The cleaning agent may be in a separate pouch; see, e.g., U.S. Pat. No. 6,037,319 or U.S. Publ. No. 2006/0223736.

The period of time may be measured in seconds or minutes, and may depend, for example, on a particular washing cycle, or on the temperature of the water, the degree of bleaching required, or on the number of layers of the pouch.

The pouch may include any suitable amount of the bleaching agent or composition comprising a bleaching agent. The amount may depend, for example, on whether the washing machine is a regular or high efficiency machine, on the size of the washing machine, on the volume of water, or on the degree of bleaching required. For example, larger volumes of water may require a higher amount of the bleaching agent or composition comprising a bleaching agent to achieve bleaching action, and smaller volumes of water may require a lesser amount. A composition comprising a bleaching agent be any composition described or exemplified herein. The bleaching agent may be any bleaching agent, including those described or exemplified herein.

In some aspects, the methods are for cleaning laundry. Generally, the methods comprise contacting laundry with a volume of water comprising one or more cleaning agents and/or one or more fabric softening agents, for a period of time sufficient to clean the laundry, optionally agitating the laundry in the water to facilitate the cleaning, optionally rinsing the laundry with a volume of water to remove the one or more cleaning agents and/or one or more fabric softening agents from the laundry, adding a pouch containing a bleaching agent or composition comprising a bleaching agent to the water such that exposure of the pouch to the water for a sufficient period of time causes the pouch to dissolve in the water, thereby releasing the contents of the pouch into the water and creating a bleach water solution, contacting the laundry with the bleach water solution for a period of time sufficient to bleach the laundry, optionally agitating the laundry in the bleach water solution to facilitate the bleaching, and rinsing the laundry with a volume of water to remove the bleach water solution from the laundry. The pouch preferably has a single layer, with the internal surface of the layer contacting the bleaching agent or composition thereof, and the external surface of the layer comprising the outer surface of the pouch. The method may further comprise drying the laundry.

Each period of time may be measured in seconds or minutes, and may depend, for example, on a particular washing cycle, or on the temperature of the water, the degree of bleaching required, or on the number of layers of the pouch.

In some aspects, the methods are for cleaning or disinfecting a surface or a material. Generally, the methods comprise adding a pouch containing a bleaching agent or composition comprising a bleaching agent to a volume of water such that exposure of the pouch to the water for a sufficient period of time causes the pouch to dissolve in the water, thereby releasing the contents of the pouch into the water and creating a bleach water solution, and contacting a surface or material with the bleach water solution for a period of time sufficient to clean the surface or material and/or for a period of time sufficient to disinfect the surface or material. The pouch preferably has a single layer, with the internal surface of the layer contacting the bleaching agent or composition thereof, and the external surface of the layer comprising the outer surface of the pouch. The time required for disinfecting a surface may be longer or shorter than the time required for cleaning a surface, but in either case, the time required may depend, in part, on the degree of dirt or stain on the surface, or the level of contamination requiring disinfecting. Contamination may be from a microorganism such as a bacteria, virus, mold, fungus, or mildew. The surface may be a counter surface, sink, floor, tile, grout, caulk/seal, toilet, shower stall, bathtub, cutting board, flatware, litter box, or other surface in need of cleaning or disinfecting.

In any of the methods, the pouch may be any pouch describe herein, and the bleaching agent may be any chlorine-based or chlorine-free, including oxidative, bleaching agent described herein. The pouch preferably has a single layer, with the internal surface of the layer contacting the bleaching agent or composition thereof, and the external surface of the layer comprising the outer surface of the pouch. The pouch is preferably not coated, although the pouch may be coated in some aspects.

The invention is not limited to the embodiments described and exemplified above, but is capable of variation and modification within the scope of the appended claims.

We claim:

1. A pouch, comprising a body comprising a water-soluble material and defining a container portion, and a bleaching agent or composition comprising a bleaching agent contained within the container portion; wherein the container portion has a single layer having an internal surface contacting the bleaching agent or composition and having an external surface which is the outermost portion of the pouch; and wherein the bleaching agent or composition has less than about 10% water by weight of the bleaching agent or composition.

2. The pouch of claim 1, wherein the water-soluble material comprises polyvinyl alcohol.

3. The pouch of claim 1, wherein the pouch does not contain any cleaning agent.

4. The pouch of claim 1, wherein the composition comprises a carrier comprising a hydrophobic vehicle.

5. The pouch of claim 4, wherein the hydrophobic vehicle comprises hexylene glycol.

6. The pouch of claim 1, wherein the composition comprises, by weight of the composition, about 0.5% to about 1.5% of a chlorine-based bleaching agent, about 6% to about 8% water, and about 90% to about 95% of a carrier.

7. The pouch of claim 1, wherein the composition comprises, by weight of the composition, about 2% to about 4% of an oxidative bleaching agent, about 5% to about 7% water, and about 88% to about 92% of a carrier.

8. The pouch of claim 1, wherein the bleaching agent or composition is contained within the container portion in a volume sufficient for one high efficiency laundry cycle.

9. The pouch of claim 1, wherein the bleaching agent or composition is contained within the container portion in a volume sufficient for one regular laundry cycle.

10. A method for cleaning and bleaching laundry, comprising contacting the laundry with a volume of water comprising one or more cleaning agents for a period of time sufficient to clean the laundry, optionally rinsing the laundry to remove the one or more cleaning agents from the laundry, adding the pouch of claim 1 to the water such that the pouch dissolves in the water thereby creating a bleach water solution, and contacting the laundry with the bleach water solution for a period of time sufficient to bleach the laundry.

11. The method of claim 10, wherein the composition comprises, by weight of the composition, about 0.5% to about 1.5% of a chlorine-based bleaching agent, about 6% to about 8% water, and about 90% to about 95% of a carrier.

12. The method of claim 10, wherein the composition comprises, by weight of the composition, about 2% to about 4% of an oxidative bleaching agent, about 5% to about 7% water, and about 88% to about 92% of a carrier.

13. The method of claim 10, further comprising rinsing the laundry to remove the bleach water solution from the laundry.

14. A laundry kit, comprising one or more pouches comprising a body comprising a water-soluble material and defining a container portion, a bleaching agent or composition comprising a bleaching agent contained within the container portion, and instructions for using the kit in a method for cleaning and bleaching laundry; wherein the container portion has a single layer having an internal surface for contacting the bleaching agent or composition and has an external surface which is an outermost portion of the pouch.

15. The laundry kit of claim 14, wherein the one or more pouches do not contain any cleaning agent.

16. The laundry kit of claim 14, wherein the one or more pouches are the pouch of claim 1.

17. The laundry kit of claim 14, wherein the composition comprises, by weight of the composition, about 0.5% to about 1.5% of a chlorine-based bleaching agent, about 6% to about 8% water, and about 90% to about 95% of a carrier.

18. The laundry kit of claim 14, wherein the composition comprises, by weight of the composition, about 2% to about 4% of an oxidative bleaching agent, about 5% to about 7% water, and about 88% to about 92% of a carrier.

19. A method for disinfecting a surface, comprising adding the pouch of claim 1 to a volume of water such that the pouch dissolves in the water thereby creating a bleach water solution, and contacting the surface with the bleach water solution for a period of time sufficient to disinfect the surface or material.

20. The method of claim 19, wherein the surface is a counter surface, sink, floor, tile, grout, caulk/seal, toilet, shower stall, bathtub, cutting board, flatware, litter box, or other surface in need of disinfecting.
